(19) United States
(12) Patent Application Publication
(10) Pub. No.: US 2011/0295134 A1
(43) Pub. Date: Dec. 1, 2011

(54) Method For ECG Screening

(75) Inventor: Thomas L. DeBauche, Cypress, TX (US)

(73) Assignee: Dr. Thomas L. DeBauche, Cypress, TX (US)

(21) Appl. No.: 13/096,743

(22) Filed: Apr. 28, 2011

Related U.S. Application Data
(60) Provisional application No. 61/328,902, filed on Apr. 28, 2010.
(60) Provisional application No. 61/349,283, filed on May 28, 2010.

(51) Int. Cl. A61B 5/0402

(52) U.S. Cl. 600/509

(57) ABSTRACT

A method for screening a selected population such as student athletes for heart disease and/or cardiac abnormalities comprises obtaining an ECG of an individual member of the population at a first location; uploading the ECG to a data storage device connected to a communications network; storing the ECG in the data storage device; subsequently downloading the stored ECG to a display device at a second location remote from the first location; interpreting and annotating the displayed ECG; and, transmitting the annotated ECG to a pre-designated recipient. The method may further include advising school officials of the results of screening so that participation in school athletics may be limited for students identified as being at risk of sudden death if full participation were allowed.

### CROSS-REFERENCE TO RELATED APPLICATIONS

This application claims the benefit of U.S. Provisional Application No. 61/328,902, filed on Apr. 28, 2010.

### STATEMENT REGARDING FEDERALLY SPONSORED RESEARCH OR DEVELOPMENT

Not Applicable

### BACKGROUND OF THE INVENTION

1. Field of the Invention

This invention relates to medical screening methods. More particularly, it relates to screening young athletes for cardiac abnormalities.

2. Description of the Related Art Including Information Disclosed Under 37 CFR 1.97 and 1.98.

Most sudden deaths in athletes are due to cardiovascular disease. Atherosclerotic coronary artery disease is the most common cause of sudden death in athletes over 35 years of age. Hypertrophic cardiomyopathy has been implicated as the principal cause of cardiac arrest in younger competitive athletes, accounting for about one third of fatal cases in the United States. The early identification of this abnormality by screening of athletes before they participate in competitive sports might prevent sudden death, but the costs associated with this strategy have prevented its widespread adoption.

An article published in The New England Journal of Medicine [Vol. 339 pp. 364-369; No. 6, August 6, 1998] by Corrado et al. and entitled “Screening for Hypertrophic Cardiomyopathy in Young Athletes” reports the study of sudden deaths among athletes and nonathletes (35 years of age or less) in the Veneto region of Italy from 1979 to 1996. The causes of sudden death in both populations were compared, and the pathological findings in the athletes were related to their clinical histories and electrocardiograms. Cardiovascular reasons for disqualification from participation in sports were investigated and follow-up was performed in a consecutive series of 33,735 young athletes who underwent pre-participation screening in Padua, Italy, during the same period. The most common causes of sudden death in athletes were arrhythmogenic right ventricular cardiomyopathy (22.4 percent), coronary atherosclerosis (18.4 percent), and anomalous origin of a coronary artery (12.2 percent). Hypertrophic cardiomyopathy caused only 1 sudden death among the athletes (2.0 percent) but caused 16 sudden deaths in the nonathletes (7.3 percent).

Electrocardiography (ECG or EKG) is a transthoracic interpretation of the electrical activity of the heart over time captured and externally recorded by skin electrodes. It is a noninvasive recording produced by an electro-cardiographic device.

The ECG works by detecting and amplifying the electrical changes on the skin that are caused when the heart muscle “depolarises” during each heart beat. At rest, each heart muscle cell has a charge across its outer wall, or cell membrane. Reducing this charge towards zero is called de-polarisation, which activates the mechanisms in the cell that cause it to contract. During each heartbeat a healthy heart will have an orderly progression of a wave of depolarisation that is triggered by the cells in the sinoatrial node, spreads out through the atrium, passes through “intrinsic conduction pathways” and then spreads all over the ventricles. This is detected as increase and decreases in the voltage between two electrodes placed on either side of the heart and which may be displayed graphically either on a screen or on paper. This display indicates the overall rhythm of the heart and weaknesses in different parts of the heart muscle.

ECG is the best way to measure and diagnose abnormal rhythms of the heart, particularly abnormal rhythms caused by damage to the conductive tissue that carries electrical signals, or abnormal rhythms caused by electrolyte imbalances. In a myocardial infarction (MI), ECG can identify if the heart muscle has been damaged in specific areas, though not all areas of the heart are covered. ECG cannot reliably measure the pumping ability of the heart, for which ultrasound-based (echocardiography) or nuclear medicine tests may be used.

The present invention provides a method which can lower the costs associated with screening a population of student athletes (or the like) prior to their participation in strenuous activities.

### BRIEF SUMMARY OF THE INVENTION

The method of the present invention provides a relatively low cost way to screen certain populations—e.g., student athletes—for cardiac abnormalities prior to their participation in activities which may overstress their hearts.

In one particular preferred embodiment, the method comprises providing ECG machines to schools; instructing school personnel on performing ECG's and on the electronic transmission of ECG traces; receiving and interpreting ECG's received from schools; notifying school officials of students needing further testing; arranging further testing for students exhibiting abnormal ECG's; and advising school officials of the results of screening so that participation in school athletics may be limited for students identified as being at risk of sudden death if full participation were allowed.

In an optional step, personnel may participate in performing or interpreting screening tests other than ECG's, such as echocardiograms, EBCT, stress testing, monitoring, etc. as necessary on clinical basis for selected individual students.

### BRIEF DESCRIPTION OF THE DRAWING FIGURES

FIG. 1 is a flow chart depicting one embodiment of the invention.

### DETAILED DESCRIPTION OF THE INVENTION

One particular preferred embodiment of the invention comprises providing ECG machines to schools and teaching school personnel how to perform ECG's on students, athletes, ADHD drug recipients, or others and how to electronically transmit the ECG data to medical personnel for interpretation.

Trained, medical personnel (preferably cardiologists) receive ECG's, interpret them and notify school officials of those students whose ECG's are within normal parameters and those for whom further testing is indicated and/or for whom participation in certain activities should be avoided until the results of further tests are available.

The method of the invention may optionally include providing, arranging, or assisting in arranging further testing for those students exhibiting abnormal ECG's.

Reviewing medical personnel may advise school officials of the results of the screening so that participation in school athletics can be limited for those students who may be at risk of sudden death if full participation were allowed.

Medical personnel may optionally participate in performing or interpreting screening tests other than ECG's, such as echocardiograms, EBCT, stress testing, monitoring, etc. as necessary on clinical basis for individual students.

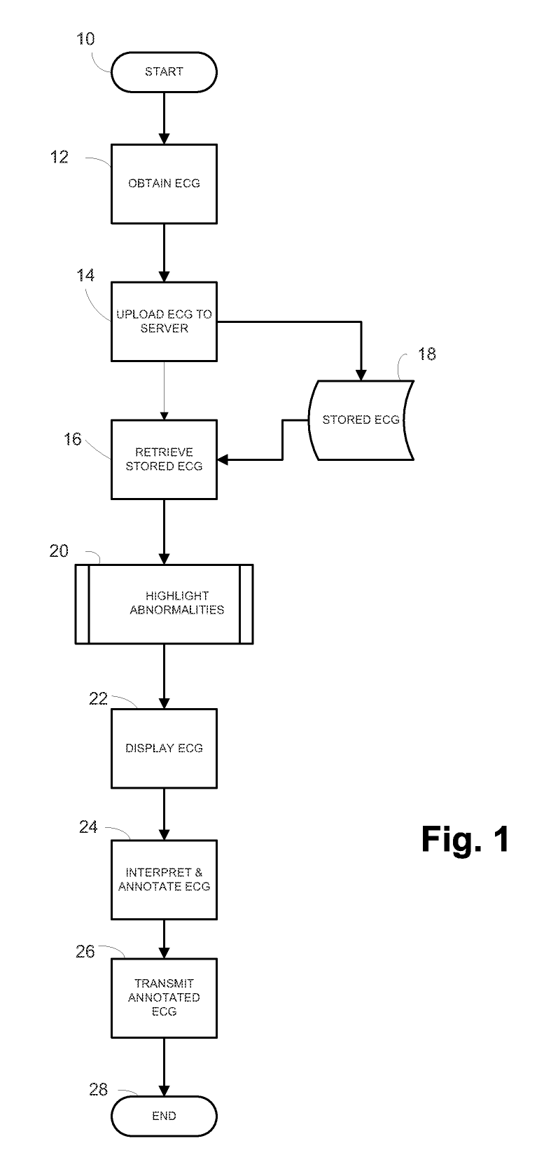

One particular preferred embodiment of the invention is illustrated in flowchart form in FIG. 1. The process begins at 10 at a location that may be remote from the location where steps 16 through 26 are performed. At step 12, an individual is connected to an ECG machine and an electrocardiogram is recorded. The ECG may be a conventional 12-lead ECG. If the output of the ECG machine is digital, the ECG may be directly uploaded to a networked server or similar storage device at 14. If the output of the ECG machine is in analog format, the waveform(s) may first be digitized and then uploaded to the server at step 14. At this point in the process, the digitized ECG of the individual is stored in a network-accessible device 18 together with identifying information for the individual tested.

Step 16 of the process may be performed at any time subsequent to the storage of the ECG in networked storage 18. In certain embodiments of the invention, storage device 18 may automatically generate and a send a message —e.g., an e-mail message—to one or more designated persons that a new ECG has been stored and is ready for interpretation. The stored ECG is downloaded from the server at step 16 by the reviewer who may be specially trained in interpreting ECG's. The reviewer may be a cardiologist and the ECG may be displayed (at step 22) on his or her personal computer which is in network communication with server 18.

Optional step 20 may be an automated process for detecting and highlighting abnormalities in the downloaded ECG. For example, a processor under software control may perform pattern recognition on the ECG and/or measure and report the duration and/or magnitude of certain identifiable segments of the ECG—e.g. the QT segment or the ST segment. In certain embodiments, this automated process may report or graphically highlight those portions of the ECG which fall outside of pre-selected “normal” limits.

In the illustrated embodiment, the reviewing physician interprets (step 24) the ECG that was displayed at step 22. Step 24 may include annotation of the ECG by the reviewing medial professional. In certain embodiments, this annotation may consist of categorizing the ECG into pre-defined categories. For example, the pre-defined categories may be: 1) “normal”—no abnormalities found, no restrictions on participation in activities; 2) “additional tests recommended”—obtain an echocardiogram or other indicated tests before allowing participation; and, 3) “alert”—possible significant heart defects indicated, stop participation.

At step 26, the annotated ECG may be transmitted electronically to selected recipients (e.g., by e-mail) and/or uploaded to a storage device (which may be storage device 18) for subsequent retrieval by authorized persons.

The process may terminate at 28 or the reviewer may select another ECG for downloading and interpretation. It will be appreciated by those skilled in the art that the illustrated process is not limited to ECG's but rather may be applied to other diagnostic tests which may benefit from remote interpretation and annotation. For example, if, as a result of abnormalities noted in an individual's ECG, an echocardiogram is obtained, those results may be similarly processed according to the illustrated method.

The method may also include collecting demographic data relating to the individuals in the population being tested and correlating the demographic data with the ECG results.

The following funding options are among those that may be considered in implementing the method of the present invention:
Charge schools for the ECG machines, or lease them (if not donated).
Charge schools for each ECG interpretation.
Apply for grant money for coverage of these and other costs.
Seek sponsorship from sports, civic, or other corporate or community organizations to underwrite the costs.

It will be appreciated by those skilled in the art that the method of the present invention is not limited to the screening of student athletes in schools. Rather, any population may be selected for screening at any convenient facility—e.g., community centers, sports complexes, YMCA facilities, churches, etc. as well as commercial enterprises.

Although the invention has been described in detail with reference to certain preferred embodiments, variations and modifications exist within the scope and spirit of the invention as described and defined in the following claims.

What is claimed is:

1. A method for screening a population comprising:
obtaining an ECG of an individual member of the population at a first location;
uploading the ECG to a data storage device connected to a communications network;
storing the ECG in the data storage device;
subsequently downloading the stored ECG to a display device at a second location remote from the first location;
interpreting the displayed ECG;
transmitting the interpreted ECG to a pre-designated recipient.

2. A method as recited in claim 1 wherein the population is a group of student athletes.

3. A method as recited in claim 1 wherein the population is a group of persons diagnosed as having attention deficit hyperactivity disorder.

4. A method as recited in claim 1 wherein the first location is a school.

5. A method as recited in claim 1 wherein the data storage device is a server.

6. A method as recited in claim 1 wherein the communications network is the Internet.

7. A method as recited in claim 1 wherein the display device is a personal computer.

8. A method as recited in claim 1 wherein the interpreting is performed by a cardiologist.

9. A method as recited in claim 1 wherein the interpreting comprises categorizing the ECG into pre-defined categories.

10. A method as recited in claim 1 wherein the pre-designated recipient is a school official.

11. A method as recited in claim 1 further comprising digitizing the ECG prior to uploading the ECG to the storage device.

12. A method as recited in claim 1 further comprising notifying at least one interpreter when an ECG is uploaded to the storage device.

13. A method as recited in claim 1 further comprising automatically identifying potential abnormalities in the ECG.

14. A method as recited in claim 13 wherein identifying comprises graphically highlighting a potential abnormality on a displayed ECG.

15. A method as recited in claim 1 further comprising annotating the displayed ECG.

16. A method as recited in claim 15 wherein annotating the displayed ECG comprises categorizing the ECG into pre-defined categories.

17. A method as recited in claim 16 wherein the pre-defined categories comprise a first category for individuals whose ECG exhibit no significant abnormalities and a second category for individuals whose ECG exhibit one or more abnormalities.

18. A method for screening a population comprising:
obtaining an ECG of an individual member of the population at a first location;
uploading the ECG to a data storage device connected to a communications network;
storing the ECG in the data storage device;
subsequently downloading the stored ECG to a display device at a second location remote from the first location;
annotating the displayed ECG;
uploading the annotated ECG to a storage device connected to a communications network.

19. A method for screening student athletes comprising:
providing ECG machines to schools;
instructing school personnel on performing ECG's and on the electronic transmission of ECG traces;
receiving and interpreting ECG's received from schools;
identifying to school officials those screened students identified as being at risk of sudden death if participation in school athletics were allowed.

20. A method as recited in claim 19 further comprising notifying school officials of students needing further testing.
